(19) United States
(12) Patent Application Publication
(10) Pub. No.: US 2020/0069288 A1
(43) Pub. Date: Mar. 5, 2020

(54) ULTRASOUND PROBE

(71) Applicant: OLYMPUS CORPORATION, Tokyo (JP)

(72) Inventor: Yuichi SHIGA, Tokyo (JP)

(73) Assignee: OLYMPUS CORPORATION, Tokyo (JP)

(21) Appl. No.: 16/669,628

(22) Filed: Oct. 31, 2019

Related U.S. Application Data
(63) Continuation of application No. PCT/JP2017/019666, filed on May 26, 2017.

(51) Int. Cl. A61B 8/00; A61B 8/12; G10K 11/35

(52) U.S. Cl. CPC A61B 8/4461; A61B 8/12; A61B 8/445; G10K 11/352; A61B 8/4494

(57) ABSTRACT

An ultrasound probe includes: an ultrasound transducer provided on a distal end side of an outer tube; an actuator including a movable portion configured to perform a reciprocating motion; and a conversion mechanism. The conversion mechanism is arranged in the longitudinal direction of the outer tube in series with the actuator in the longitudinal direction, arranged in parallel to the ultrasound transducer in the longitudinal direction, and configured to convert the reciprocating motion of the movable portion to a swing motion of the ultrasound transducer. A working point in the mechanism is positioned off a center axis of the outer tube and a center axis of driving force of the reciprocating motion in the movable portion, and the center axis of the driving force of the reciprocating motion in the movable portion is displaced to the working point side by a predetermined distance relative to the center axis.

### CROSS REFERENCE TO RELATED APPLICATION

This application is a continuation application of PCT/JP2017/019666 filed on May 26, 2017, the entire contents of which are incorporated herein by this reference.

### BACKGROUND OF THE INVENTION

### 1. Field of the Invention

The present invention relates to an ultrasound probe, and in particular relates to an ultrasound probe of a mechanical scanning system.

### 2. Description of the Related Art

Conventionally, an ultrasound imaging device has been widely used in a medical field or the like, and an ultrasound probe that is inserted into a body cavity or into a blood vessel to observe internal organs has been developed.

An ultrasound transducer is provided on a distal end of a probe, and for a scanning system of the ultrasound transducer, either an electronic scanning system or a mechanical scanning system is used. When it is taken into consideration that the probe itself is to be inserted into a body cavity or the like, it is preferable that a diameter of the probe is small.

For example, Japanese Patent Application Laid-Open Publication No. 63-145640 discloses an ultrasound probe that achieves the mechanical scanning system by swinging an ultrasound transducer around a predetermined axis.

A turning member mounted with the ultrasound transducer is arranged inside a distal end portion of the ultrasound probe. In order to make the turning member turn around a turning axis, a rigid operation wire moves back and forth in an axial direction of the ultrasound probe. A distal end portion of the operation wire is fixed to one end of the turning member.

A point of the turning member to which the distal end portion of the operation wire is fixed becomes a working point when the turning member is driven to swing, and the turning member swings around the turning axis. By the turning member swinging around the turning axis, the ultrasound transducer swings around the turning axis, and mechanical scanning is performed. The working point is positioned on an opposite side of an ultrasound transducer mounting portion of the turning member relative to the turning axis.

In order to efficiently supply force by back-and-forth movements of the operation wire to the working point, it is preferable that the working point is positioned on a center axis of the operation wire which is a driving member.

### SUMMARY OF THE INVENTION

An ultrasound probe of one aspect of the present invention includes: an outer tube to be inserted into a body; an ultrasound transducer provided on a distal end side of the outer tube and supported swingably within a plane in a longitudinal direction of the outer tube; an actuator including a movable portion configured to perform a reciprocating motion in the longitudinal direction of the outer tube; and a conversion mechanism arranged in the longitudinal direction of the outer tube in series with the actuator in the longitudinal direction, arranged in parallel to the ultrasound transducer in the longitudinal direction, and configured to convert the reciprocating motion of the movable portion to a swing motion of the ultrasound transducer, in which a working point in the conversion mechanism of force by the reciprocating motion of the actuator is positioned off a center axis of the outer tube and a center axis of driving force of the reciprocating motion in the movable portion, and the center axis of the driving force of the reciprocating motion in the movable portion is displaced to the working point side by a predetermined distance relative to the center axis of the outer tube.

### BRIEF DESCRIPTION OF THE DRAWINGS

FIG. 1 is an external view of an ultrasound probe according to an embodiment of the present invention;

FIG. 2 is a perspective view of a distal end portion 2 of an ultrasound probe 1 according to the embodiment of the present invention;

FIG. 3 is a perspective view of an actuator of an ultrasound transducer disposed inside the distal end portion 2, according to the embodiment of the present invention;

FIG. 4 is a front view of an actuator 22 according to the embodiment of the present invention;

FIG. 5 is a left side view of the actuator 22 according to the embodiment of the present invention;

FIG. 6 is a right side view of the actuator 22 according to the embodiment of the present invention;

FIG. 7 is a sectional view of the actuator 22 according to the embodiment of the present invention;

FIG. 8 is a sectional view of the distal end portion 2 of the ultrasound probe 1 when an ultrasound transducer 21 is at an intermediate position when the ultrasound transducer 21 is not swinging, according to the embodiment of the present invention;

FIG. 9 is a sectional perspective view of the distal end portion 2 when the ultrasound transducer 21 is at the intermediate position when the ultrasound transducer 21 is not swinging, according to the embodiment of the present invention;

FIG. 10 is a perspective view of a linking member 32b according to the embodiment of the present invention;

FIG. 11 is a plan view of the linking member 32b according to the embodiment of the present invention;

FIG. 12 is a diagram for explaining an arrangement state of a transducer mounting portion 33 and the linking member 32b, according to the embodiment of the present invention;

FIG. 13 is a sectional view of the distal end portion 2 when an ultrasound transmission/reception surface 21a of the ultrasound transducer 21 is inclined to a distal end side of the distal end portion 2, according to the embodiment of the present invention;

FIG. 14 is a sectional view of the distal end portion 2 when the ultrasound transmission/reception surface 21a of the ultrasound transducer 21 is inclined to a proximal end side of the distal end portion 2, according to the embodiment of the present invention;

FIG. 15 is a diagram for explaining a direction of driving force given to an extension portion 33b by the linking member 32b, according to the embodiment of the present invention; and

FIG. 16 is a diagram for explaining a difference in reaction force due to a moment applied to the actuator 22 between a case where a center axis of the driving force is located on a center axis O of an outer tube 22t and a case where the center axis of the driving force is displaced by a predetermined distance d.

### DETAILED DESCRIPTION OF THE PREFERRED EMBODIMENT

Hereinafter, the embodiment of the present invention will be described using drawings.

(Overall Structure)

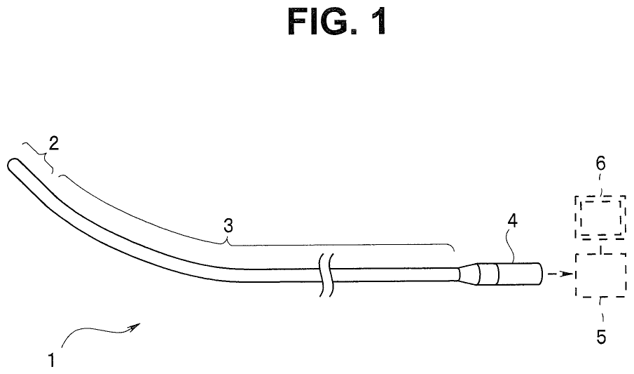

FIG. 1 is an external view of an ultrasound probe. An ultrasound probe 1 has an elongated shape, and includes a distal end portion 2, a flexible tube portion 3 and a connection portion 4 from a distal end.

The ultrasound probe 1 is a probe of a mechanical scanning system having the elongated shape that can be inserted into a narrow lumen such as a blood vessel, pulmonary peripheral site, and a pancreas and biliary system. The ultrasound probe 1 includes an elongated and flexible resin outer tube 2t, and a distal end portion 2 fixed to a distal end of the outer tube 2t. A diameter of the ultrasound probe 1 is, for example, 2 to 5 mm. The distal end portion 2 incorporates an ultrasound transducer of the mechanical scanning system, as described later.

The flexible tube portion 3 is formed of a flexible tube member, and a signal line or the like is inserted inside.

The connection portion 4 is connected to an ultrasound observation device 5, as illustrated by a dotted line. To the ultrasound observation device 5, a monitor 6 is connected. The ultrasound observation device 5 includes an image processing portion configured to process an output signal of the ultrasound probe 1, generate an ultrasound image and output the ultrasound image to the monitor 6.

The ultrasound observation device 5 includes a drive circuit device configured to electronically drive the ultrasound transducer inside the distal end portion 2, and an image generation portion configured to generate the ultrasound image inside a subject based on a signal from the ultrasound transducer. The ultrasound observation device 5 outputs an image signal of the generated ultrasound image to the monitor 6 which is a display device, and the ultrasound image is displayed on the monitor 6.

A user can insert the ultrasound probe 1 into a treatment instrument insertion channel of an endoscope (not illustrated) in endoscopy for example, and view the ultrasound image of the subject displayed on the monitor 6 to perform an examination.

(Configuration of Distal End Portion of Ultrasound Probe)

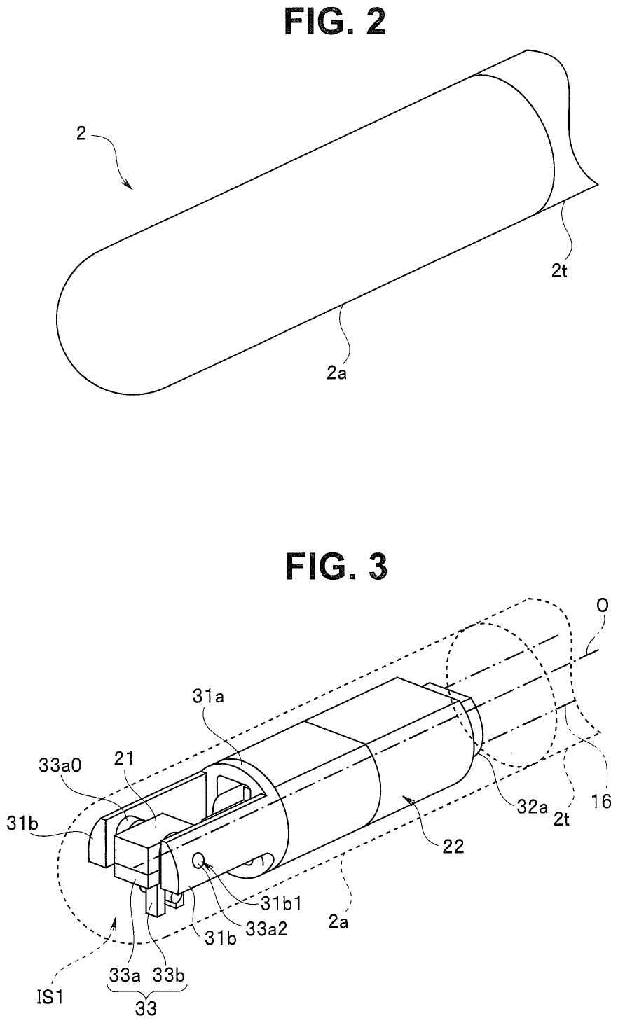

FIG. 2 is a perspective view of the distal end portion 2 of the ultrasound probe 1. FIG. 3 is a perspective view of an actuator of the ultrasound transducer disposed inside the distal end portion 2.

As illustrated in FIG. 2, the distal end portion 2 of the ultrasound probe 1 includes a resin housing 2a. The outer tube 2t to be inserted into a body and the housing 2a provided on a distal end side of the outer tube 2t are configured to be inserted into the body of the subject.

The housing 2a has a cylindrical shape, and a distal end side part of the housing 2a is formed into a semispherical shape. A center axis of the housing 2a having the cylindrical shape coincides with a center axis O of the ultrasound probe 1.

On an inner side of the housing 2a, as illustrated in FIG. 3, an internal space IS1 configured to house an ultrasound transducer 21 is provided, and the ultrasound transducer 21 is made swingable within a predetermined angle range around a predetermined axis inside the internal space IS1 by an actuator 22 disposed inside the distal end portion 2. The ultrasound transducer 21 is provided on the distal end side of the outer tube 2t, and is supported swingably within a plane in a longitudinal direction of the outer tube 2t. The ultrasound transducer 21 is an element formed of a single plate of a roughly rectangular outer shape.

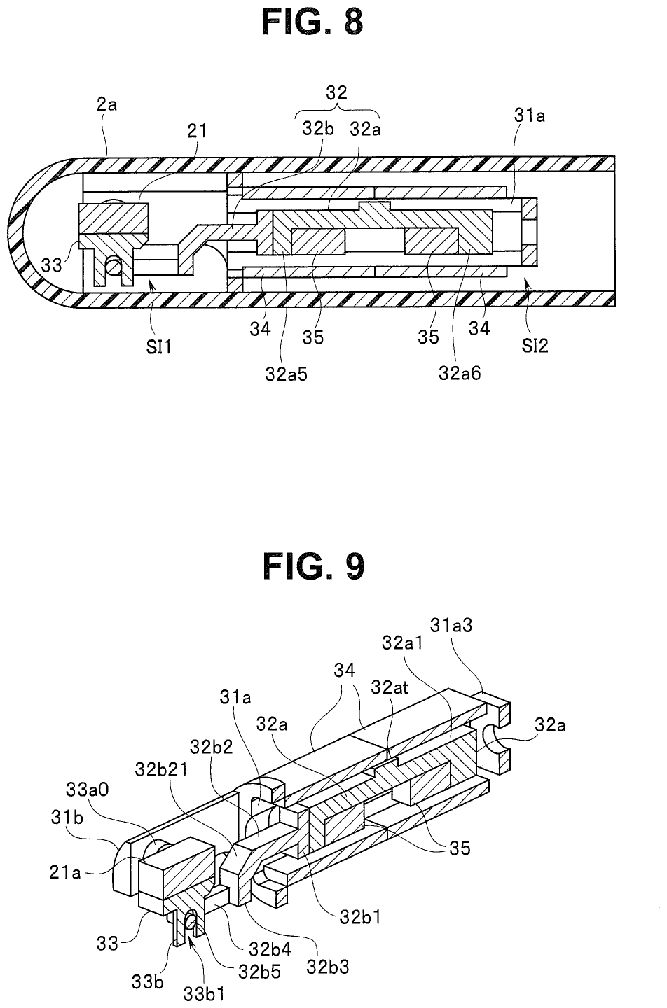

The housing 2a includes the internal space IS1 configured to house the ultrasound transducer 21 and a conversion mechanism 33X (to be described later) on the distal end side, and an internal space IS2 configured to house the actuator 22 on a proximal end side (see FIG. 8). The actuator 22 has a shape elongated in a direction of the axis O of the ultrasound probe 1. A center axis of the actuator 22 coincides with the center axis of the housing 2a.

As illustrated by two-dot chain lines in FIG. 3, a plurality of signal lines 16 for the ultrasound transducer 21 and the actuator 22 extend from the proximal end side of the actuator 22, and are inserted into the outer tube 2t and the flexible tube portion 3.

(Configuration of Actuator)

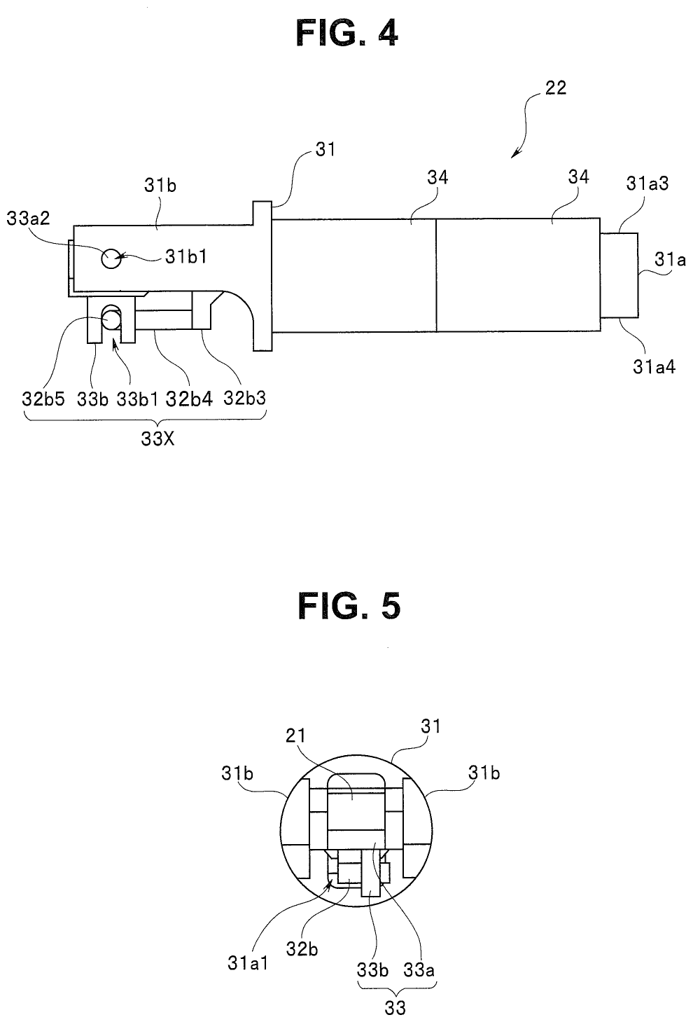

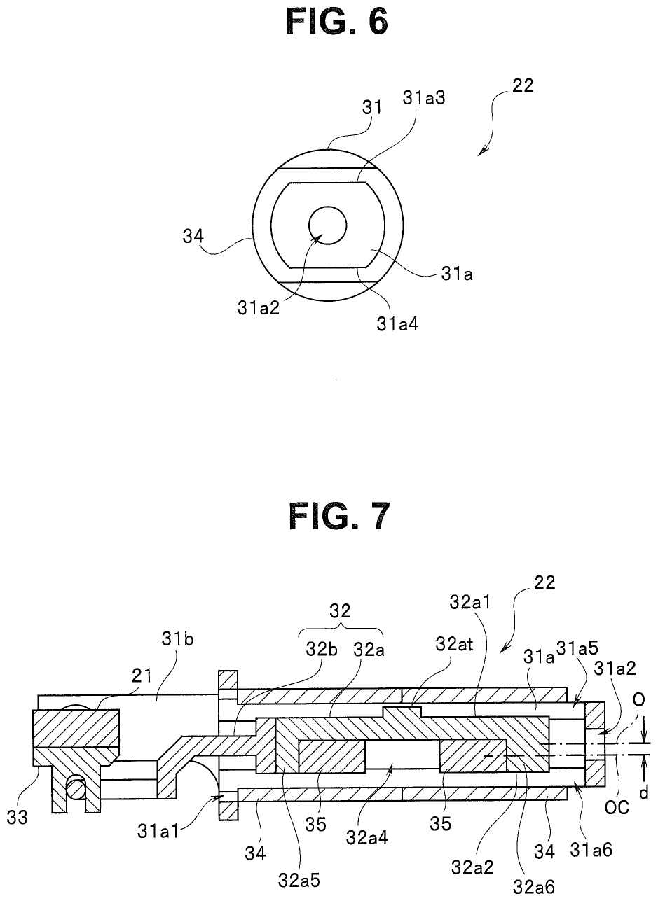

FIG. 4 is a front view of the actuator 22. FIG. 4 is a diagram of a view orthogonal to the axis of the distal end portion 2. FIG. 5 is a left side view of the actuator 22. FIG. 6 is a right side view of the actuator 22. FIG. 5 illustrates a side face when viewing the actuator 22 disposed inside the distal end portion 2 from the distal end side, and FIG. 6 illustrates a side face when viewing the actuator 22 disposed inside the distal end portion 2 from the proximal end side. FIG. 7 is a sectional view of the actuator 22. FIG. 8 is a sectional view of the distal end portion 2 of the ultrasound probe 1 when the ultrasound transducer 21 is at an intermediate position when the ultrasound transducer 21 is not swinging. FIG. 9 is a sectional perspective view of the distal end portion 2 when the ultrasound transducer 21 is at the intermediate position when the ultrasound transducer 21 is not swinging.

As described above, the actuator 22 is disposed inside the internal space IS2 of the distal end portion 2 of the ultrasound probe 1 (see FIG. 8), and is a drive unit configured to make the ultrasound transducer 21 swing within the predetermined angle range around the predetermined axis. Here, the actuator 22 is an electromagnetic drive unit using a moving magnet type voice coil motor which is an electromagnetic actuator. The voice coil motor includes a coil and a magnet.

Note that in the present embodiment, the moving magnet type voice coil motor in which a movable portion includes the magnet is used for the actuator 22, but a moving coil type voice coil motor may be used as the actuator 22.

The actuator 22 which is the electromagnetic actuator includes a frame member 31, a movable portion 32 disposed inside the frame member 31, and a transducer mounting portion 33 disposed on the distal end side of the movable portion 32 and mounted with the ultrasound transducer 21. The movable portion 32 performs a reciprocating motion in a longitudinal direction of the outer tube 2t.

As illustrated in FIG. 3 and FIG. 7, the actuator 22 is disposed inside the ultrasound probe 1 such that a longitudinal axis of the actuator 22 coincides with the center axis O of the ultrasound probe 1 and the longitudinal axis of the actuator 22 becomes parallel to the center axis O.

As illustrated in FIG. 4 to FIG. 6, the frame member 31 includes a cylindrical portion 31a and two arms 31b. As illustrated in FIG. 3 and FIG. 4, the two arms 31b are provided so as to extend in a distal end direction from the distal end of the cylindrical portion 31a in parallel to each other. On each arm 31b, a hole 31b1 through which a shaft portion 33a2 to be described later is to be inserted is formed.

As illustrated in FIG. 5 and FIG. 7, a hole 31a1 is formed on the distal end side of the cylindrical portion 31a. As illustrated in FIG. 6 and FIG. 7, a hole 31a2 is also formed on the proximal end side of the cylindrical portion 31a. In other words, the actuator 22 includes the frame member 31 configured to house the movable portion 32 inside, and the hole 31a2 is formed on an opposite side of the conversion mechanism 33X to be described later of the frame member 31. The hole 31a2 is a hole for injecting liquid, oil in this case, into the housing 2a of the distal end portion 2 for impedance matching when manufacturing the ultrasound probe 1. The oil injected from the hole 31a2 passes through the hole 31a1 and enters the internal space IS1.

As illustrated in FIG. 6, an upper side face 31a3 and a lower side face 31a4 of the cylindrical portion 31a have a flat surface. As illustrated in FIG. 7, a through groove 31a5 extending in an axial direction of the cylindrical portion 31a is formed on the upper side face 31a3 of the cylindrical portion 31a, and a through groove 31a6 extending along the axis of the cylindrical portion 31a is also formed on the lower side face 31a4 of the cylindrical portion 31a.

On an outer peripheral surface of the cylindrical portion 31a, two coil portions 34 formed by winding a coil wire are provided.

Then, the movable portion 32 includes a column member 32a and a linking member 32b which extends in the axial direction of the distal end portion 2 from the distal end side of the column member 32a.

On an upper surface and a lower surface of the column member 32a, two plane portions 32a1 and 32a2 formed by being cut along the axis of the cylindrical portion 31a are provided respectively. The plane portion 32a1 is in parallel to the upper side face 31a3 of the cylindrical portion 31a, and the plane portion 32a2 is in parallel to the lower side face 31a4 of the cylindrical portion 31a.

On the plane portion 32a1, a protruding portion 32at is formed. The protruding portion 32at has such a height that a part of the protruding portion 32at enters the through groove 31a5, and restricts rotation around the axis of the movable portion 32.

As described later, the column member 32a is movable along the axis of the distal end portion 2 inside the cylindrical portion 31a in a state where the protruding portion 32at enters the through groove 31a5.

Further, the column member 32a includes an elongated recessed portion 32a4 formed along the axis of the column member 32a. The recessed portion 32a4 is, as illustrated in FIG. 7, formed on a lower side of the column member 32a. Inside the recessed portion 32a4, two permanent magnets 35 are arranged along the axis of the column member 32a. Each permanent magnet 35 has a rectangular parallelepiped shape, and is arranged inside the recessed portion 32a4 with an N pole and an S pole in a predetermined direction so that a magnetizing direction is a direction orthogonal to the axis of the cylindrical portion 31a.

As illustrated in FIG. 7, the respective coil portions 34 and the respective permanent magnets 35 have such a shape that a dimension in a direction of the reciprocating motion is longer than a dimension in an orthogonal direction of the reciprocating motion of the movable portion 32. Thus, space efficiency of the distal end portion 2 is excellent.

In addition, as illustrated in FIG. 7, since the two coil portions 34 formed by the coil wire are arranged in series along the center axis O of the distal end portion 2 and the two permanent magnets 35 are also arranged in series along the center axis O of the distal end portion 2, the space efficiency of the distal end portion 2 is excellent.

As described later, while the column member 32a moves back and forth along the center axis O of the distal end portion 2, as illustrated in FIG. 7, a longitudinal axis OC of the two permanent magnets 35 is displaced from the center axis O of the distal end portion 2 by a distance d and positioned.

Note that the column member 32a is formed of a ferromagnetic body. Here, for the movable portion 32, by using a member of the ferromagnetic body as a material of the column member 32a that holds the two permanent magnets 35, magnetic resistance is reduced, output efficiency of a magnetic circuit is improved, and force to move the movable portion 32 is increased.

Further, drive of the actuator is affected by disturbance acceleration accompanying disturbance (back and forth movement or bending of the probe by an operation of an operator for example) and variation among individuals. Therefore, a swing motion of the ultrasound transducer is not smoothly performed and there are more than a few risks of image degradation such as blurring of an ultrasound image. For such a problem, it is desirable to execute feedback control to the actuator; however, since a sensor needs to be separately provided in that case, a diameter of the device is increased.

In the present embodiment, by using the ferromagnetic body for the movable portion 32, change of inductance of the coil to position change of the movable portion 32 is enlarged so that a speed or a position of the movable portion 32 can be estimated without using a sensor and so-called sensorless control can be applied.

In the coil portions 34, induced electromotive force is generated by movement of the movable portion 32. In addition, by a current flowing to the coil portions 34, self-induced electromotive force is also generated. By detecting the self-induced electromotive force, the above-described sensorless control can be performed. The part of the ferromagnetic body of the movable portion 32 is arranged on the inner side of the two coil portions 34 so that the change of magnitude of the current flowing to the coil portions 34 generated based on the induced electromotive force can detect an end moving position of the movable portion 32. For accurate position control of the movable portion 32, it is preferable that a length in the axial direction of the column member 32a of the ferromagnetic body is formed to be longer than the length of the two coil portions 34 in the direction of the center axis O of the distal end portion 2.

As a result, based on the magnitude of the current flowing to the coil portions 34, the position of the movable portion 32 in the actuator 22 can be detected.

Also, as illustrated in FIG. 7, since the two permanent magnets 35 are disposed inside the recessed portion 32a4 of the column member 32a, in the case where the column member 32a is formed of the ferromagnetic body, the two permanent magnets 35 are disposed between a distal end portion 32a5 and a proximal end portion 32a6 of the column member 32a. In other words, the member of the ferromagnetic body is provided on both sides that are the distal end side and the proximal end side of the two permanent magnets 35. Thus, influence of a magnetic field from outside in the direction of the center axis O of the distal end portion 2 can be shielded, and even when the magnetic body is present at front and back in the axial direction of the distal end portion 2, magnetic force drawn by the magnetic body is reduced, and the output efficiency of the actuator 22 is excellent.

Since the coil wire is wound on an outer side of the cylindrical portion 31a of the frame member 31, an outer shape of the coil portions 34 includes two plane portions 34a and 34b along the upper side face 31a3 and the lower side face 31a4 of the cylindrical portion 31a. In other words, a part of the outer shape of the two coil portions 34 includes the plane portions.

The respective coil portions 34 and the respective permanent magnets 35 are disposed so that the two plane portions of the rectangular parallelepiped permanent magnets 35 become parallel to the two plane portions 34a and 34b.

The two coil portions 34 and the two permanent magnets 35 of the movable portion 32 form the moving magnet type voice coil motor.

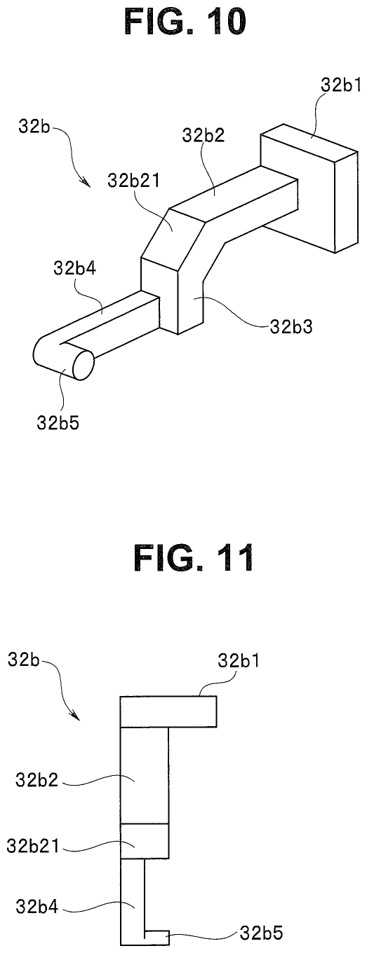

FIG. 10 is a perspective view of the linking member 32b. FIG. 11 is a plan view of the linking member 32b.

As illustrated in FIG. 10, the linking member 32b includes a connection portion 32b1, a first extension portion 32b2 extending from the connection portion 32b1 to the distal end side in parallel to the center axis O, a bending portion 32b3 bent in a direction orthogonal to the center axis O at the distal end portion of the first extension portion 32b2, a second extension portion 32b4 extending from one end of the bending portion 32b3 to the distal end side in parallel to the center axis O, and a pin portion 32b5 protruding in the direction orthogonal to the center axis O at the second extension portion 32b4.

In other words, the movable portion 32 includes the linking member 32b extending toward the distal end side. The pin portion 32b5 of the linking member 32b engages with a through slit 33b1 of an extension portion 33b of a fixation portion 33a described later.

The connection portion 32b1 is fixed and connected with an adhesive material to the distal end portion 32a5 of the column member 32a moving back and forth in the direction of the center axis O.

An inclined portion 32b21 is provided on the distal end portion of the first extension portion 32b2, and the bending portion 32b3 is connected with the distal end portion of the first extension portion 32b2 via the inclined portion 32b21. The inclined portion 32b21 is provided for not allowing the linking member 32b to restrict a swing range of the ultrasound transducer 21 which is made to swing inside the internal space IS1 and making it difficult for the linking member 32b to be in contact with other members inside the internal space IS1 or IS2 when the linking member 32b moves to the proximal end side.

As illustrated in FIG. 3, the transducer mounting portion 33 includes the fixation portion 33a where the ultrasound transducer 21 of the roughly rectangular outer shape is mounted and fixed, and the extension portion 33b extending from the fixation portion 33a. In other words, the fixation portion 33a is a mounting portion configured to mount the ultrasound transducer 21. The extension portion 33b extends to the opposite side of the fixation portion 33a relative to a swing axis of the swing motion of the ultrasound transducer 21. The fixation portion 33a includes two sidewall portions 33a0. The respective sidewall portions 33a0 include the two shaft portions 33a2. The two shaft portions 33a2 are coaxially formed so as to protrude from the two sidewall portions 33a0 in directions opposite to each other.

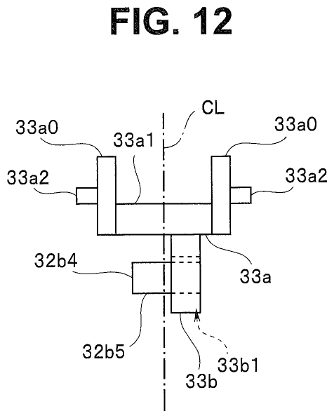

FIG. 12 is a diagram for explaining an arrangement state of the transducer mounting portion 33 and the linking member 32b. FIG. 12 is a diagram viewing the linking member 32b and the transducer mounting portion 33 from the distal end side of the distal end portion 2. As illustrated in FIG. 12, the fixation portion 33a includes a plane portion 33a1 for fixing the ultrasound transducer 21 of the single plate by fixing means such as the adhesive material. The ultrasound transducer 21 is disposed between the two sidewall portions 33a0.

The two shaft portions 33a2 protruding in the directions opposite to each other are inserted to the two holes 31b1 of the two arms 31b such that the fixation portion 33a can be turned around the shaft portions 33a2.

The extension portion 33b extends in the direction orthogonal to the plane portion 33a1 from the surface on the opposite side of the plane portion 33a1. As illustrated in FIG. 4, the extension portion 33b includes the through slit 33b1. The pin portion 32b5 of the linking member 32b enters the through slit 33b1.

More specifically, the through slit 33b1 is formed such that the fixation portion 33a is supported by the two arms 31b turnably around the axis of the two shaft portions 33a2, and the pin portion 32b5 can move inside the through slit 33b1 when the fixation portion 33a is turned around the axis of the two shaft portions 33a2.

In addition, as illustrated in FIG. 10 to FIG. 12, the first extension portion 32b2 and the second extension portion 32b4 of the linking member 32b are displaced from a plane CL in parallel to a vertical direction passing through the center axis O of the distal end portion 2 and extend in the distal end direction. Simultaneously, the extension portion 33b of the transducer mounting portion 33 also extends from a position displaced from the plane CL in parallel to the vertical direction passing through the center axis O of the distal end portion 2. Thus, since a thickness in the direction orthogonal to the plane CL of the linking member 32b and the extension portion 33b can be reduced, the linking member 32b and the extension portion 33b are not easily brought into contact with an inner wall of the housing 2a of the distal end portion 2.

In other words, the extension portion 33b of the transducer mounting portion 33 and the linking member 32b are arranged so as to hold the plane CL that passes through the center axis O of the outer tube 2t and is orthogonal to the axis of the swing motion of the ultrasound transducer 21 between the extension portion 33b and the linking member 32b. The axis of the swing motion of the ultrasound transducer 21 is the axis of the two shaft portions 33a2.

When the linking member 32b moves back and forth within a predetermined range along the axis of the distal end portion 2, the pin portion 32b5 also moves in back and forth directions along the axis of the distal end portion 2. The reciprocating motion of the pin portion 32b5 moves the extension portion 33b that engages with the pin portion 32b5, and generates the swing motion that makes the fixation portion 33a swing around the axis of the two shaft portions 33a2.

Note that the linking member 32b extending from the column member 32a is formed of a non-magnetic body or a weak magnetic body. In other words, the part on the side of the conversion mechanism 33X described later of the movable portion 32 is the non-magnetic body or the weak magnetic body. When the linking member 32b is the ferromagnetic body, since inductance change becomes asymmetrical when performing the above-described sensorless control, there is a risk that the position of the movable portion 32 cannot be accurately detected. Therefore, the linking member 32b is formed of the non-magnetic body or the weak magnetic body to prevent deterioration of accuracy of position detection.

As above, inside the two coil portions 34 formed of the coil wire wound around the axis of the distal end portion 2 of the ultrasound probe 1, the two permanent magnets 35 are disposed such that N pole and the S pole are in the predetermined direction along the axis of the distal end portion 2.

(Action)

Next, operations of the ultrasound transducer 21 and the actuator 22 inside the distal end portion 2 of the ultrasound probe 1 will be described.

By the two coil portions 34 and the two permanent magnets 35, the moving magnet type voice coil motor is formed. By changing the direction of the current made to flow to the respective coil wires of the two coil portions 34, a moving direction of the two permanent magnets 35 along the axis of the distal end portion 2 can be controlled.

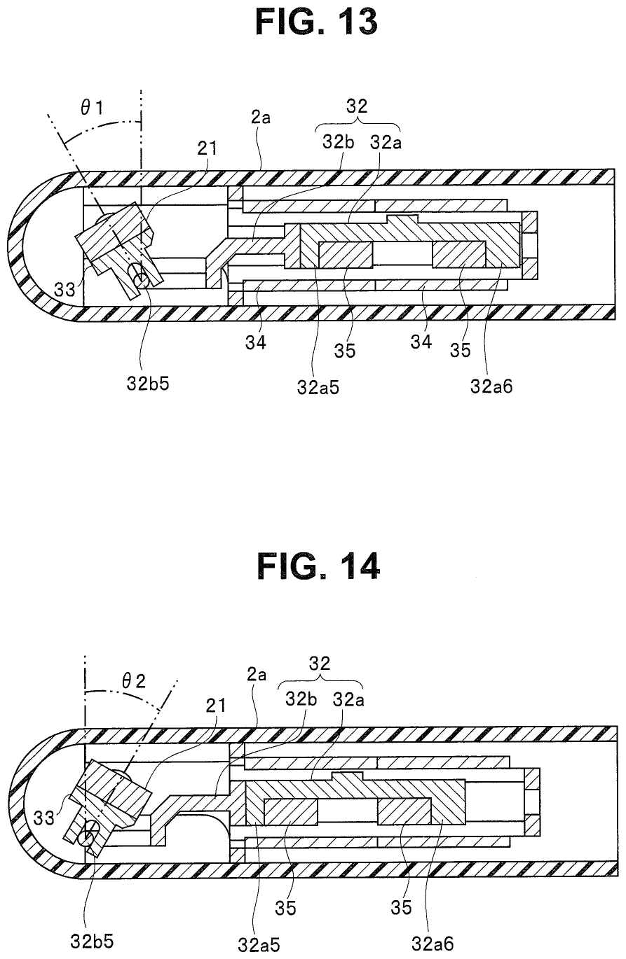

FIG. 13 is a sectional view of the distal end portion 2 when an ultrasound transmission/reception surface 21a of the ultrasound transducer 21 is inclined to the distal end side of the distal end portion 2. FIG. 14 is a sectional view of the distal end portion 2 when the ultrasound transmission/reception surface 21a of the ultrasound transducer 21 is inclined to the proximal end side of the distal end portion 2.

The conversion mechanism 33X generates the swing motion of the ultrasound transducer 21 by the linking member 32b moving the extension portion 33b of the transducer mounting portion 33 back and forth in the longitudinal direction. More specifically, when the current in a first direction is made to flow to the respective coil wires of the two coil portions 34, by a principle of the moving magnet type voice coil motor, the movable portion 32 moves to the proximal end side and comes into contact with the inner wall on the proximal end side of the cylindrical portion 31a, as illustrated in FIG. 13. At the time, the ultrasound transmission/reception surface 21a of the ultrasound transducer 21 turns to the distal end side. As illustrated in FIG. 13, a normal direction of the ultrasound transmission/reception surface 21a is inclined to the distal end side by θ1 relative to the direction orthogonal to the axis of the distal end portion 2.

When the current in a second direction opposite to the first direction is made to flow to the respective coil wires of the two coil portions 34, by the principle of the moving magnet type voice coil motor, the movable portion 32 moves to the distal end side and comes into contact with the inner wall on the distal end side of the cylindrical portion 31a, as illustrated in FIG. 14. At the time, the ultrasound transmission/reception surface 21a of the ultrasound transducer 21 turns to the proximal end side. As illustrated in FIG. 14, the normal direction of the ultrasound transmission/reception surface 21a is inclined to the proximal end side by θ2 relative to the axis orthogonal to the axis of the distal end portion 2.

Thus, by alternately changing the direction of making the current flow, the ultrasound transducer 21 is made to swing in the range of an angle (θ1+θ2) around the shaft portion 33a2.

Thus, the movable portion 32 including the pin portion 32b5 and the extension portion 33b including the through slit 33b1 that engages with the pin portion 32b5 configure the conversion mechanism 33X that converts the reciprocating motion of the movable portion 32 to the swing motion of the ultrasound transducer 21. The conversion mechanism 33X is arranged in the longitudinal direction of the outer tube 2t in series with the actuator 22 in the longitudinal direction, arranged in parallel to the ultrasound transducer 21 in the longitudinal direction, and configured to convert the reciprocating motion of the movable portion 32 to the swing motion of the ultrasound transducer 21.

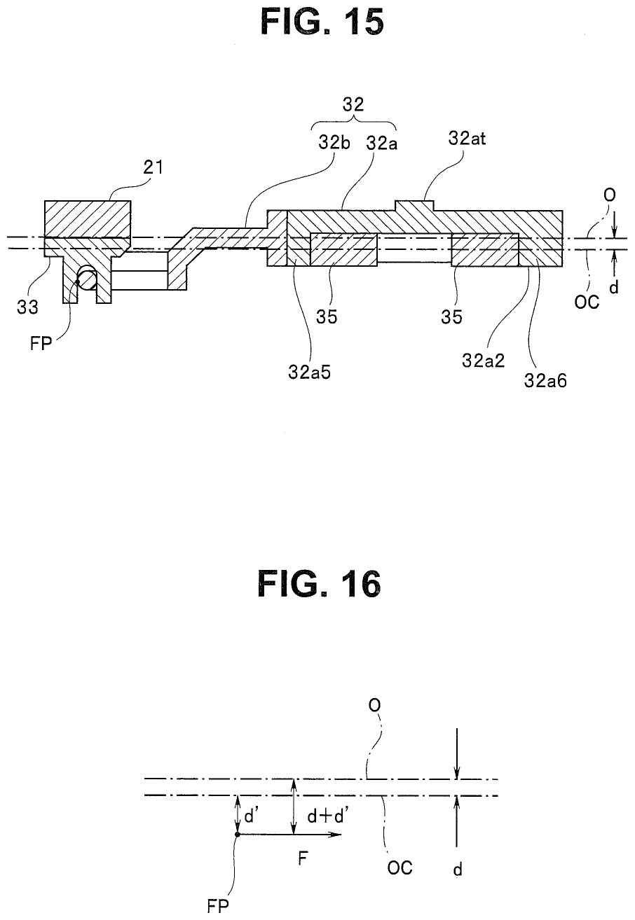

FIG. 15 is a diagram for explaining a direction of the driving force given to the extension portion 33b by the linking member 32b.

As shown in FIG. 15, a working point FP at which force is applied to the extension portion 33b when the linking member 32b moves back and forth is positioned off the center axis O of the outer tube 2t and a center axis OC of the driving force of the reciprocating motion in the movable portion 32.

However, the longitudinal axis OC of the two permanent magnets 35, which generate driving force in the actuator 22, is displaced to the working point FP side with respect to the center axis O of the outer tube 2t. Specifically, the working point FP in the conversion mechanism 33X of force by the reciprocating motion of the actuator 22 is positioned off the center axis O of the outer tube 2t, and the longitudinal axis OC of the permanent magnets 35, which is the center axis of the driving force of the reciprocating motion in the movable portion 32, is displaced to the working point FP side with respect to the center axis O by a predetermined distance d.

Therefore, reaction force, which is generated at the working point FP due to a moment applied to the actuator 22, becomes smaller than reaction force in the case where the center axis of the driving force F1 of the actuator 22 is located on the center axis O.

FIG. 16 is a diagram for explaining a difference in the reaction force due to the moment applied to the actuator 22 between the case where the center axis of the driving force F1 is located on the center axis O of the outer tube 2t and the case where the center axis of the driving force F1 is displaced by a predetermined distance d. When the center axis of the driving force F1 is located on the center axis O of the outer tube 2t, the reaction force due to the moment applied to the actuator 22 is represented by F×(d+d′). On the other hand, when the center axis of the driving force F1 is located on OC displaced from the center axis O by the predetermined distance d, the reaction force due to the moment applied to the actuator 22 is represented by F×d′, and the reaction force becomes smaller than the above-described value.

If the reaction force, which is generated at the working point FP due to the moment applied to the actuator 22, is small, effective conversion of the driving force F1 to the swing motion is possible. If the reaction force, which is generated at the working portion FP due to the moment applied to the actuator 22, is large, strong contact occurs between the inner wall of the cylindrical portion 31a of the frame member 31 and the column member 32a of the movable portion 32. Such strong contact causes large frictional force between the inner wall and the column member. The inner wall of the cylindrical portion 31a is shaved off by the friction, and the shavings created by the inner wall of the cylindrical portion 31a being shaved off float in the liquid in the internal space IS1, which may lead a possibility that a clear ultrasound image cannot be obtained. That is, as described above, the reaction force, which is generated at the working point FP due to the moment applied to the actuator 22, is made small, to thereby enable the durability of the conversion mechanism 33X to be increased.

In the above described embodiment, the center axis of the actuator 22 coincides with the center axis O of the ultrasound probe 1. Such a configuration does not create what is called a dead space in the distal end portion 2. As a result, the outer diameter of the actuator 22 can be increased, which enables the driving force of the actuator 22 to be also increased.

Furthermore, since the point GP where the driving force F1 of the actuator 22 is generated is displaced to the working point FP side with respect to the center axis O, the reaction force, which is generated at the working point FP due to the moment applied to the actuator 22, becomes small, which enables effective transmission of the driving force F1.

As above, according to the above-described embodiment, the ultrasound probe that suppresses increase of the diameter of the probe including a swing mechanism which swings the ultrasound transducer and suppresses decline of force conversion efficiency when converting a back and forth motion of a driving member to the swing motion around a turning axis of a turning member can be provided.

The present invention is not limited to the above-described embodiment, and can be variously changed and modified or the like without departing from the gist of the present invention.

What is claimed is:

1. An ultrasound probe comprising:
an outer tube to be inserted into a body;
an ultrasound transducer provided on a distal end side of the outer tube and supported swingably within a plane in a longitudinal direction of the outer tube;
an actuator including a movable portion configured to perform a reciprocating motion in the longitudinal direction of the outer tube; and
a conversion mechanism arranged in the longitudinal direction of the outer tube in series with the actuator in the longitudinal direction, arranged in parallel to the ultrasound transducer in the longitudinal direction, and configured to convert the reciprocating motion of the movable portion to a swing motion of the ultrasound transducer, in which a working point in the conversion mechanism of force by the reciprocating motion of the actuator is positioned off a center axis of the outer tube and a center axis of driving force of the reciprocating motion in the movable portion, and the center axis of the driving force of the reciprocating motion in the movable portion is displaced to the working point side by a predetermined distance relative to the center axis of the outer tube.

2. The ultrasound probe according to claim 1,
wherein the actuator is an electromagnetic actuator.

3. The ultrasound probe according to claim 2,
wherein the electromagnetic actuator is a voice coil motor including a coil and a magnet.

4. The ultrasound probe according to claim 3,
wherein the coil or the magnet is in such a shape that a dimension in a direction of the reciprocating motion is longer than a dimension in an orthogonal direction of the reciprocating motion.

5. The ultrasound probe according to claim 4,
wherein the voice coil motor is of a moving magnet type in which the movable portion includes the magnet.

6. The ultrasound probe according to claim 5,
wherein the movable portion includes a ferromagnetic body member.

7. The ultrasound probe according to claim 6,
wherein the ferromagnetic body member is provided on both sides that are a distal end side and a proximal end side of the magnet.

8. The ultrasound probe according to claim 7,
wherein a part on the conversion mechanism side of the movable portion is a non-magnetic body or a weak magnetic body.

9. The ultrasound probe according to claim 1,
wherein the actuator includes a frame member configured to house the movable portion inside and a hole is formed on a side opposite to the conversion mechanism of the frame member.

10. The ultrasound probe according to claim 1, comprising
an ultrasound transducer mounting portion including a mounting portion configured to mount the ultrasound transducer and a first extension portion extending toward an opposite side of the mounting portion relative to a swing axis of the swing motion,
wherein the movable portion includes a second extension portion extending toward the distal end side and engaged with the first extension portion,
the conversion mechanism generates the swing motion of the ultrasound transducer by the second extension portion causing the first extension portion to move back and forth in the longitudinal direction, and
the first extension portion and the second extension portion are arranged so as to hold a plane that passes through the center axis of the outer tube and is orthogonal to an axis of the swing motion of the ultrasound transducer between the first extension portion and the second extension portion.

11. The ultrasound probe according to claim 1, comprising
a housing provided on the distal end side of the outer tube and including a first internal space configured to house the ultrasound transducer and the conversion mechanism and a second internal space configured to house the actuator.
